(19) United States
(12) Patent Application Publication
(10) Pub. No.: US 2002/0151832 A1
(43) Pub. Date: Oct. 17, 2002

(54) Composite finger flexion glove

(76) Inventor: Roy D. Wedge JR., Midland, MI (US)

Correspondence Address: Roy D. Wedge, Jr., 4008 Cruz Dr., Midland, MI, 48642, US

(21) Appl. No.: 10/071,739

(22) Filed: Feb. 8, 2002

Related U.S. Application Data
(60) Provisional application No. 60/268,985, filed on Feb. 15, 2001.

(51) Int. Cl. A61F 5/00; A63B 23/16

(52) U.S. Cl. 602/22; 482/47

(57) ABSTRACT

The present invention relates to a finger rehabilitation device that applies composite finger flexion forces to the fingers of a human hand for the purpose of achieving improved hand function. The glove portion of the device is worn on a human hand and has means for securable attachment to the hand. The glove has attachment tabs at its fingertips for removable attachment to a crossbar that may be rotated around its longitudinal axis to apply forces of bending to each of the selected fingers. The rotational force of the crossbar is generated by an outrigger that may be manually positioned as desired. The orientation of the outrigger in reference to the longitudinal axis of the crossbar may be selectively fixed at one end of the crossbar, providing ease of force adjustment while the device is worn. The selected outrigger position is further maintained by an elastic or non-elastic attachment to the wearer's wrist.

### CROSS-REFERENCE TO RELATED APPLICATIONS

This application is entitled to the benefit of Provisional Patent Application Serial No. 60/268,985, filed Feb. 15, 2001.

### BACKGROUND—FIELD OF INVENTION

This invention relates to the use of external devices to facilitate the rehabilitation of an injured human hand.

### BACKGROUND—DESCRIPTION OF PRIOR ART

The complexity of the human hand makes it susceptible to stiffness and decreased mobility following an injury. Occupational therapists and physical therapists provide stretching, exercises, and activity to help the injured individual regain range of motion of the fingers. Improved range of motion of the fingers ultimately results in improved strength and functional use of the injured hand. Each of the fingers of a human hand has three joints, a distal interphalangeal (DIP) joint that is the joint closest to the fingertip, a proximal interphalangeal (PIP) joint that is the joint next closest to the fingertip and the metacarpalphalangeal (MP) joint at the connection of the finger to the hand. All of the finger joints must flex simultaneously in order to achieve full grasp of an item. For example, the fingers of each hand must wrap around a golf club handle in a pattern of simultaneous flexion of the MP, PIP, and DIP joints in order to create a firm grip. The grip becomes weaker if one of the joints is not flexed. Imagine trying to swing a golf club with flexion of the PIP and DIP joints of the fingers but not the MP joints. Simultaneous flexion of the finger joints is also required for screwdriver use, firmly gripping the steering wheel of a vehicle, using a large spoon to stir cake batter, and on and on. Simultaneous flexion of the MP, PIP, and DIP joints of a finger is called composite finger flexion. An individual displays full composite flexion of the fingers when the hand is in a fisted position.

Over the span of decades there have been numerous devices used to facilitate the rehabilitation of hand injuries. These devices fall into the category of rehabilitation orthotics and are generally referred to as splints. Hand therapy specialists routinely use hand and/or finger splints to position the injured hand or finger in a way that: provides protection, prevents further deformity, or facilitates increased range of motion. Splints that hold a body part in a set position are referred to as static splints. Static splints are generally used to provide protection or prevent further deformity. Splints that apply a force to create or allow movement of a body part are called dynamic or static progressive splints. For example, a dynamic splint may stabilize the wrist and hand while using a rubber band to place a dynamic force on one or more fingers. Dynamic splints are often used to facilitate increased functional movement by applying a force to the stiff or injured part over an extended period of time. In Clinical Mechanics of the Hand, Dr. Paul Brand and Anne Hollister have identified the importance of gentle continuous stretch to lengthening of tissue in the injured hand.¹ The force is usually applied by an elastic component of a spring or rubber band. There are several existing devices that focus on increasing finger flexion, but they are limited by a number of factors:

(a) There is a “finger flexion glove” that is currently available in most hand rehabilitation catalogs. This device can be described as a lightweight glove with a wrist strap and an eyelet on the end of each finger. Rubber bands are attached to the eyelets in the fingertips of the glove and then to a hook on the glove wrist strap. The tension of the rubber bands creates an elastic tension that pulls on the glove, bending the fingers into flexion. The “finger flexion glove” is effective in facilitating partial flexion of the fingers, but does not accomplish full composite flexion of all of the finger joints. The arrow in prior art FIG. 8 identifies the direction of force at the end of a finger in a “finger flexion glove”. The direction of the force and the orientation of the hand prevent simultaneous bending of all of the finger joints. The ability to adjust the tension or force provided by the “finger flexion glove” is limited. The only way to adjust the forces on the fingers is to change the size of the rubber band or change the location of the rubber band attachment.

(b) Another arrangement is referred to as the “Biodynamic™ flexion/extension system (U.S. Pat. No. 5,413,554)”. This system is most often used in conjunction with a static thermoplastic wrist splint fitted to the wearer's forearm. The forces of this system are applied to a line that connects to two individual finger straps. The fingers are flexed as the line is pulled and attached to the hand or wrist. This arrangement is useful, but requires two separately attached straps for each finger and is somewhat difficult to adjust. There is also a mechanical disadvantage to the arrangement, which requires a significant force to pull the finger into full composite flexion. The arrows in prior art FIG. 7 show the direction of pull on the finger straps.

(c) Another arrangement is primarily a static progressive system including a circumferential wrist wrap and other attachments described in U.S. Pat. No. 5,447,490. It is useful to note that the three types of finger strapping arrangements provide a static flexing force to the fingers, not a dynamic one. One set of straps is used to bend the MP and PIP joints simultaneously. The second set of straps is used to bend the MP, PIP and DIP joints simultaneously. The third set of straps are used in conjunction with rubber band attachments to allow for exercise of the fingers, but do not provide a dynamic composite bending force. The disadvantages of this arrangement are evident. The system is complex and requires the use of many individual pieces for each finger. The bending forces applied to the fingers are static, the only dynamic component of the system is provided for exercise. The purchase of several different systems for each phase of finger movement can be costly.

(d) None of the above examples has a system that applies a dynamic force of simultaneous composite finger flexion to each of the fingers of an injured hand.

(e) The adjustment of each of the above methods is limited and cumbersome.

(f) None of the above devices provide a support component to the volar (palm side) surface of a finger or fingers allowing them to wrap around a bar in a normal gripping pattern. FIG. 9 shows the volar placement of a crossbar at the end of a finger. As the crossbar is rotated by the forces shown as arrows, the finger will progressively wrap around it as the rehabilitation continues.

(g) Other devices usually involve the use of a static splint and arrangements of finger loops, lines, and adjustment mechanisms that are cumbersome and difficult to don and doff.

### SUMMARY

In accordance with the present invention a composite finger flexion glove is used to apply a dynamic force of composite finger flexion to each of the fingers of an injured hand in a way that is easily adjustable, is easily donned and doffed, and facilitates rehabilitation of an injured hand to a normal grip pattern.

### Objects and Advantages

The present invention has been specifically designed to provide dynamic forces that act on the joints of the fingers of the hand in a composite flexion pattern. It is important to apply force to all of the finger joints in a composite pattern to recreate full functional use of the fingers of the injured hand. The arrangement consists of a glove that has attached loop fastener tabs at the end of each of the four finger locations. The loop fastener tabs are matched with a crossbar that has hook fastener material attached to it in a manner typically used for mated loop and hook fasteners in the industry. The crossbar is shaped to promote comfortable placement of the fingertips of the hand and provide secure attachment of the glove to the crossbar. The fingers of the hand are curled or wrapped around the crossbar by its' torsion forces and are held there by the glove. The crossbar provides a volar support component at each of the selected fingers. The crossbar has an outrigger attached to it. The outrigger transfers the force of the elastic component to the crossbar that in turn applies the force to fingers of a wearer's hand. One end of the elastic component is attached to the outrigger and the other end is attached at the wrist of the wearer. The outrigger transmits the dynamic force of the elastic component into a torsion force that is applied to the crossbar at one of its ends. The torque is transmitted to the MP, PIP, and DIP joints of the hand through the combined action of the glove and the crossbar. The arrangement is adjustable in three ways; the placement of the loop fastener tabs on the crossbar may be individually adjusted for each finger, the orientation of the outrigger may be selectively fixed at its connection to the crossbar, and the size of the elastic component may be varied. The wearer may easily make adjustments without the use of special tools and has the ability to easily refine the torque adjustment of the crossbar for comfort. The entire arrangement is easily donned and doffed. The wearer puts the glove on, adjusts the outrigger to the desired position and attaches the elastic component to the wrist or hand. Accordingly, besides the objects and advantages of the composite finger flexion glove described above, several objects and advantages of the present invention are:

(a) To provide the use of a composite finger flexion glove that applies a dynamic force of simultaneous composite finger flexion to each selected finger in a way that is easily adjustable.

(b) To provide the use of a composite finger flexion glove that applies a dynamic force of simultaneous composite finger flexion to each selected finger in a way that is easily donned and doffed and is easily adjustable.

(c) To provide the use of a composite finger flexion glove that applies a dynamic force of composite finger flexion to each selected finger in a way that is easily donned and doffed, is easily adjustable, and does not require the use of several individual components that are awkward and cause increased expense.

(d) To provide the use of a composite finger flexion glove that applies a dynamic force of simultaneous composite finger flexion to each selected finger of an injured hand.

(e) To provide the use of a composite finger flexion glove that provides for adjustment in three different ways, and is easily adjusted while worn.

(f) To provide the use of a composite finger flexion glove that applies a volar support component to each of the selected fingers as the dynamic composite flexion force is applied.

(g) To provide the use of a composite finger flexion glove that can be easily donned and doffed by placing the glove on an injured hand and adjusting the other components to the desired force.

### DRAWING FIGURES

FIG. 1 shows a view of the composite finger flexion glove when worn.

FIG. 2 shows a view of the glove, tabs attached to the glove fingertips, and the wrist strap.

FIG. 3 shows a view of the crossbar and outrigger.

FIG. 4 shows an end view of the crossbar where one end of the outrigger fits for adjustment.

FIG. 5 shows a cross sectional view of the crossbar where the ends of the outrigger fit into place.

FIG. 6 shows a cross sectional view of the crossbar in the center section, where no hole is required for the outrigger placement.

FIG. 7 shows a view of prior art Biodynamic™ flexion/extension system.

FIG. 8 shows a view of a finger in a prior art “finger flexion glove”.

FIG. 9 shows a view of the composite finger flexion glove crossbar at the end of a fingertip.

### REFERENCE NUMERALS IN DRAWINGS

Reference Numerals In Drawings

| | | | |
|---|---|---|---|
| 11 | Elastic Component | 20 | Glove |
| 21a, 21b, 21c, 2d | Loop fastener tabs | 22 | Wrist strap |
| 30 | Outrigger | 40 | Crossbar |
| 41 | Notch | 42 | Hole |
| 43 | Hook fastener attachment | | |

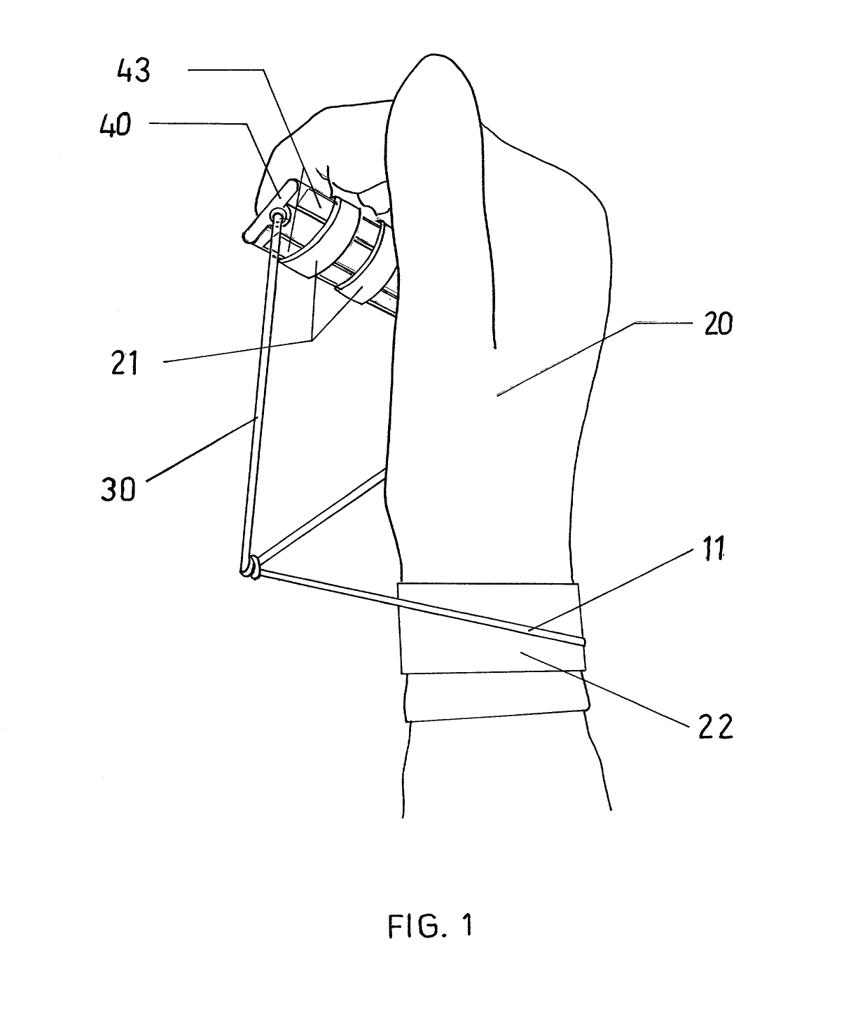

### DESCRIPTION—FIGS. 1-7

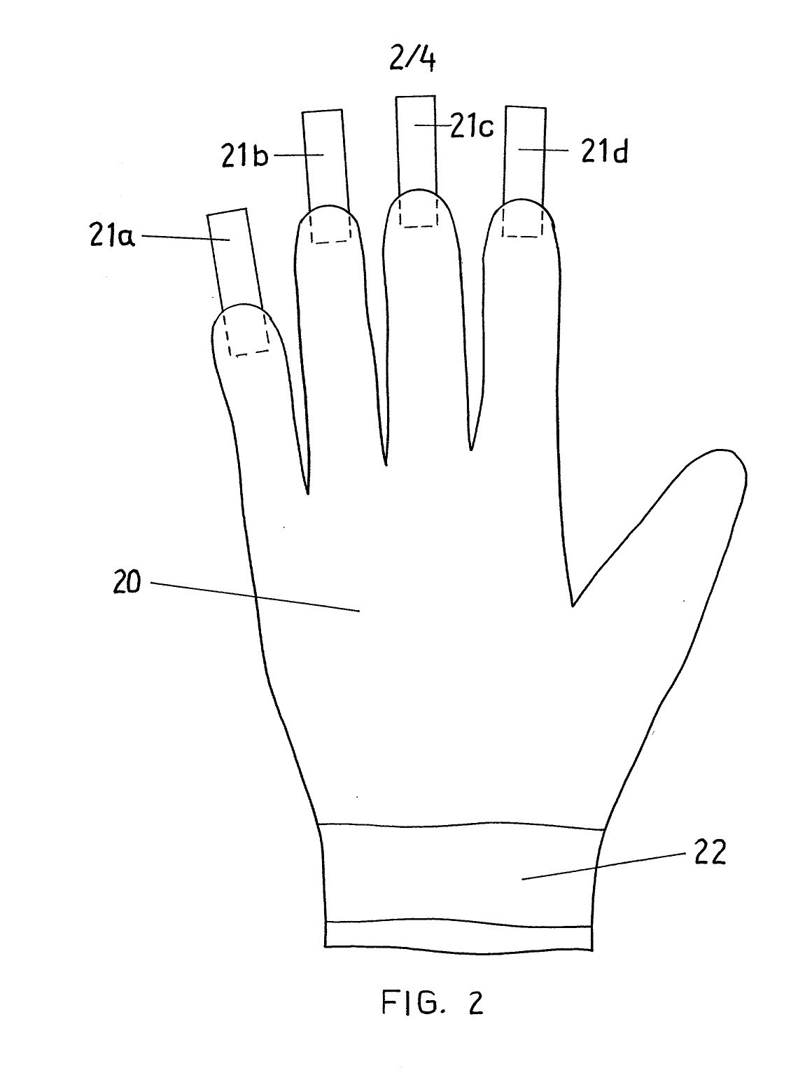

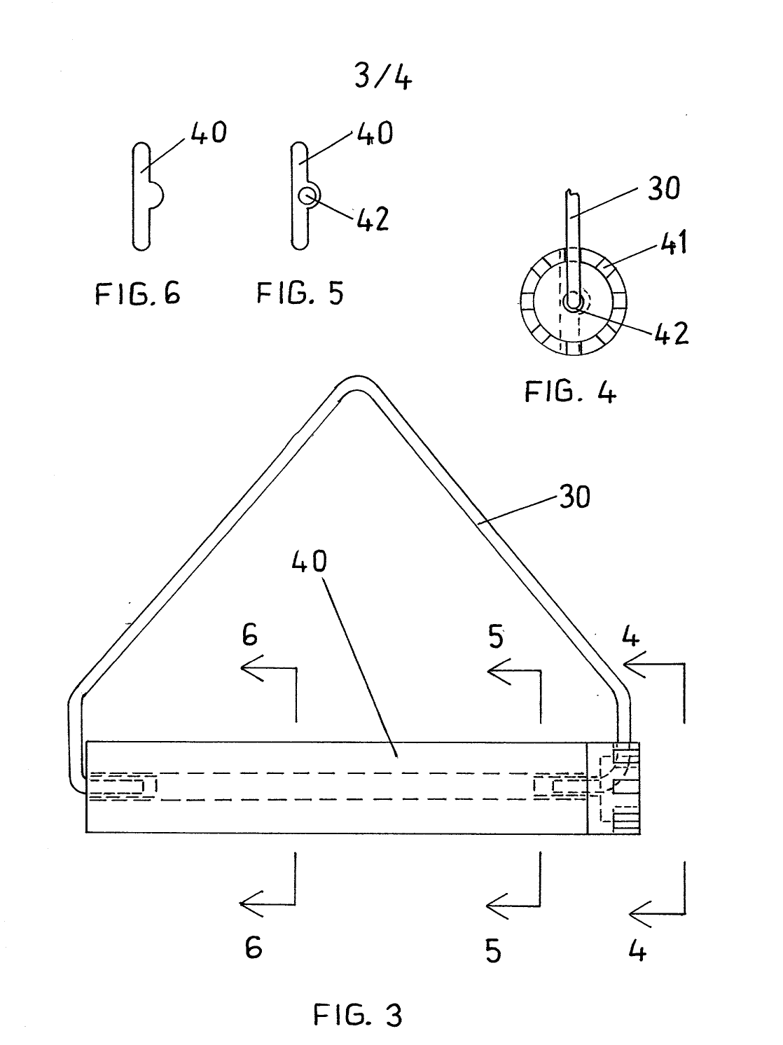

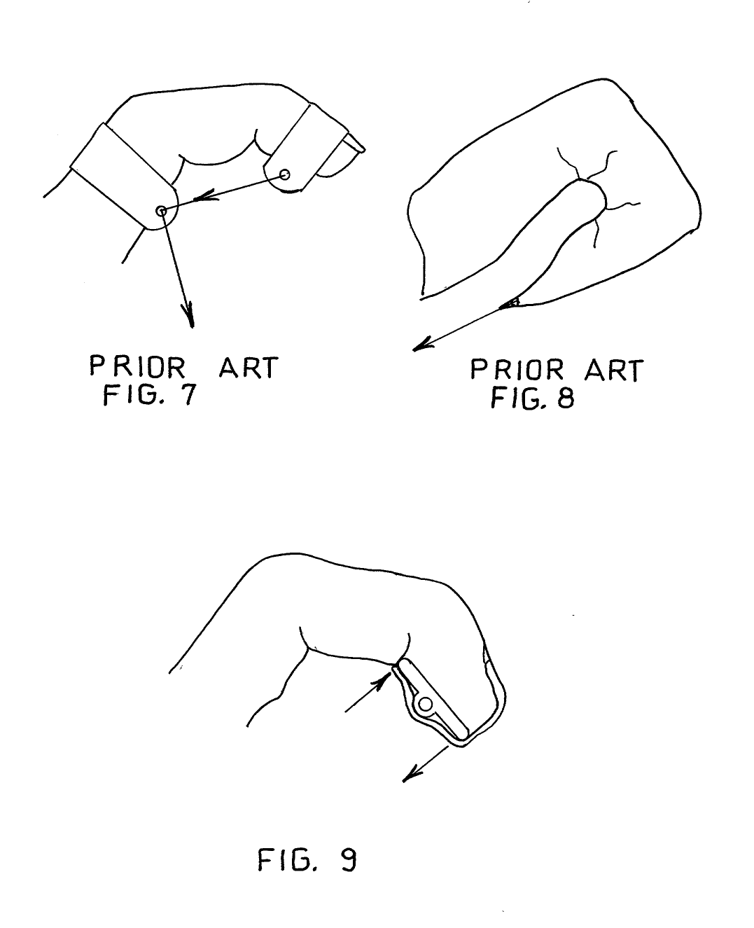

A preferred embodiment of the composite finger flexion glove is shown in FIG. 1. The glove 20 is made of material strong enough to transfer the required force to the fingers while flexible enough to allow finger movement into the desired position. Loop fastener tabs 21a, 21b, 21c, and 21d are attached to the ends of the finger portions of the glove 20. The crossbar 40 has hook fastener material 43 attached to both sides for interface with the loop fastener tabs 21a, 21b, 21c, and 21d. When the glove 20 is worn and the tabs 21a, 21b, 21c, and 21d are attached to the crossbar 40, the rotation of the crossbar 40 creates a flexion force that is transferred to the finger joints of the hand. The rotation of the crossbar 40 is achieved by adjusting the outrigger rod 30 into the desired position and attaching the elastic component 11 from the outrigger rod 30 thence around the hand or wrist. FIG. 2 shows the glove 20, attached loop fastener tabs 21a, 21b, 21c, 21d, and the wrist strap 22. FIG. 3 shows the shape of the outrigger 30 that is made of material strong enough to apply the desired torsion to the crossbar 40 yet flexible enough to allow easy placement into the ends of the crossbar 40. FIG. 1 shows the anticipated placement of the elastic component 11 that can be of varied size and shape depending upon the desired tension. FIG. 3 shows the crossbar 40 with the outrigger rod 30 inserted into holes 42 at the ends. The holes 42 (shown in FIG. 4 and FIG. 5) at each end of the cross member 40 are sized to accept the ends of the outrigger 30 and provide ample clearance to allow free rotation of the outrigger 30. FIG. 4 is an end view of the adjustable portion of crossbar 40. The notches 41 at one end of crossbar 40 are provided to selectively restrict rotation of the outrigger 30 relative to the longitudinal axis of the crossbar 40. The notches 41 are sized to accept the circular outrigger 30 for easy placement. Adjustment of the position of the outrigger 30 is accomplished by pulling it out at the notched end of the cross member 40, rotating it to one of the desired notches 41, then seating it in one of the desired notches 41.

### Advantages

From the description above, a number of advantages of the composite finger flexion glove are apparent:

(a) The device applies a dynamic force of simultaneous composite finger flexion to each of the selected fingers of an injured hand.

(b) The glove can be easily donned and doffed.

(c) The crossbar provides a volar support component to the device, facilitating the stretch of the fingers into a normal gripping pattern.

(d) There are three ways that the device may be adjusted to the needs of the wearer.

1) The tabs attached to the glove may be placed at varied locations on the crossbar.

2) Increasing or decreasing the dynamic force on the fingers may be achieved by changing the selected orientation of the outrigger in the notches at the end of the crossbar.

3) The size and type of elastic component may be changed to either decrease or increase the dynamic force applied to the outrigger.

(e) The required components are simple and can be easily manufactured.

(f) The force lever arm of the outrigger is sufficiently long to provide a generous amount of torque at the crossbar with very little applied dynamic force by the elastic component.

### OPERATION—FIGS. 1-7

The manner of use of the composite finger flexion glove is easy to understand and adaptable to the needs of the wearer. The glove 20 is attached to the crossbar 40 by the loop fastener tabs 21a, 21b, 21c, and 21d. The outrigger 30 will normally be in place at the ends of the crossbar 40. The glove 20 is placed on the injured hand and the wrist strap 22 is fastened around the wrist. The outrigger 30 is rotated to the desired position and seated in one of the desired notches 41. The elastic component 11 is normally attached to the outrigger 30 before the glove 20 is donned and then attached to the hand or wrist as the final adjustment step. To remove the composite finger flexion glove the elastic component 11 is first removed from the wrist, then the glove 20 is removed, still having the crossbar 40 and the outrigger 30 attached. The next time the composite finger flexion glove is worn it should require only minor adjustment. The dynamic force of the elastic component 11 is transferred through the outrigger 30 to the crossbar 40. The torsion force at the end of crossbar 40 is transferred to each finger's end by the tab attachments 21a, 21b, 21c, and 21d. The force at the ends of the wearer's fingers is a torsion force that is transferred to the other joints of each finger, creating composite flexion forces. The ability of the fingers to wrap around the crossbar is determined by their stiffness, as their flexibility and range of motion increase they will flex into a grip pattern around the crossbar 40.

### Conclusion, Ramifications, and Scope

The composite finger flexion glove provides a simple arrangement for the rehabilitation of the fingers of an injured hand. It is easily donned and doffed, provides three methods of adjustment, and provides a primary means of adjustment that is easily accomplished while the composite finger flexion glove is worn. It applies a dynamic composite flexion force to the fingers, has a volar support component for each finger, encourages a normal gripping pattern as fingers gradually begin to wrap around the crossbar, and is comprised of components that are easily manufactured.

Although the description above contains many specificities, these should not be construed as limiting the scope of the invention but as merely providing illustrations of some of the presently preferred embodiments of this invention. For example, the covering of the hand need not be a glove, but could be another arrangement of material that transfers the forces in a similar manner. The outrigger may be of a different form and shape than the one shown. The crossbar shape and method of transfer of the forces may be varied to accomplish the same end result. The entire crossbar and outrigger component may be made smaller to be used for an individual finger or any number of fingers. The elastic component may be replaced with a static or static progressive component.

Thus the scope of the invention should be determined by the appended claims and their legal equivalents, rather than by the examples given.

I claim:

1. A hand rehabilitation device to be worn by an individual for the purpose of improving finger flexion range of motion of an injured hand by applying simultaneous composite dynamic flexion forces to selected fingers, said hand rehabilitation device having a volar support component at the selected fingers and comprising:
(a) a glove having a back hand side, a palm side and selected fingers extending therefrom for comfortably fitting onto a hand of an individual;
(b) tabs attached to the end of each selected glove finger, said tabs to provide a means for removable attachment to a substantially rigid crossbar in order to assist with the transfer of dynamic forces to said selected fingers of an individual's hand;
(c) a substantially rigid crossbar with means to transfer a dynamic force from an outrigger to said selected fingers of an individual's hand, said crossbar having the means: to receive removable attachments of said tabs of paragraph (b) above, to receive removable attachments of an outrigger at each end, and to selectively restrict the rotation of the outrigger about the longitudinal axis of said crossbar;
(d) an outrigger with means: to transfer a dynamic force of an elastic component to a crossbar, of removable attachment to the ends of a crossbar, to allow its' selective restriction of rotation around the longitudinal axis of a crossbar, and of removable attachment to an elastic component;
(e) a elastic component with means of removable attachment to an outrigger, and means of removable attachment to an individual wearing the device.

2. The hand rehabilitation device of claim 1 wherein the tabs attached to the selected fingers of the glove are of a mating loop material and the attachment means of the crossbar is a mating hook material.

3. The hand rehabilitation device of claim 1 wherein the crossbar length is made suitable to the number of selected fingers.

4. The hand rehabilitation device of claim 1 wherein there are three means of adjustability: tabs of the selected fingers of the glove may be attached in different selected locations, the angular position of the outrigger in relation to the longitudinal axis of the crossbar may be selectively fixed, and the size of the elastic component and its' attachment point may be varied.

5. The hand rehabilitation device of claim 1 wherein the crossbar has a slotted arrangement at one end to selectively restrict outrigger rotation around the longitudinal axis of said crossbar.

6. The hand rehabilitation device of claim 1 wherein the crossbar has holes in each end to receive the ends of the outrigger.

7. The hand rehabilitation device of claim 1 wherein the outrigger is of a substantially rigid material that will transfer a dynamic force adequately to the crossbar, but also has material flexibility that allows it to be placed into the ends of the crossbar.

8. The hand rehabilitation device of claim 1 wherein the elastic component may attach to the outrigger or the wearer in any number of acceptable manners currently in use.

9. The hand rehabilitation device of claim 1 wherein there is a means of restraining the glove from sliding distally during its use.

10. The hand rehabilitation device of claim 9 wherein the restraining member is a strap securely fixed to the glove and having a mating loop and hook fastener to secure its closure around the wearer's hand or wrist.

11. The hand rehabilitation device of claim 1 wherein the elastic component may be replaced with a static or static progressive component.

12. A hand rehabilitation device to be worn by an individual for the purpose of improving finger flexion range of motion of an injured hand by applying simultaneous composite dynamic flexion forces to fingers 2, 3, 4, and 5, said hand rehabilitation device having a volar support component to the fingers and comprising:
(a) a glove having a back hand side, a palm side and five fingers extending therefrom for comfortably fitting onto a hand of an individual;
(b) tabs attached to the end of glove fingers 2, 3, 4, & 5, said tabs to provide a means for removable attachment to a substantially rigid crossbar in order to assist with the transfer of composite dynamic flexion forces to said fingers of the individual's hand;
(c) a substantially rigid crossbar with means to transfer a dynamic force from an outrigger to said fingers of an individual's hand, said crossbar having the means: to receive removable attachments of said tabs of paragraph (b) above, to receive removable attachments of an outrigger at each end, and to selectively restrict the rotation of an outrigger about the longitudinal axis of said crossbar;
(d) an outrigger with means: to transfer a dynamic force of an elastic component to a crossbar, of removable attachment to the ends of a crossbar, to allow its' selective restriction of rotation around the longitudinal axis of a crossbar, and of removable attachment to an elastic component;
(e) a elastic component with means of removable attachment to an outrigger, and means of removable attachment to an individual wearing the device.

13. The hand rehabilitation device of claim 12 wherein the tabs attached to the fingers of the glove are of a mating loop material and the attachment means of the crossbar is a mating hook material.

14. The hand rehabilitation device of claim 12 wherein the crossbar has holes at each end to receive the ends of the outrigger.

15. The hand rehabilitation device of claim 12 wherein the crossbar has a slotted arrangement at one end to selectively restrict outrigger rotation around the longitudinal axis of said crossbar.

16. The hand rehabilitation device of claim 12 wherein the outrigger is of a substantially rigid material that will transfer a dynamic force adequately to a crossbar but has material flexibility that allows it to be placed into the ends of a crossbar.

17. The hand rehabilitation device of claim 12 wherein the elastic component may attach to the outrigger or the wearer in any number of acceptable manners currently in use.

18. The hand rehabilitation device of claim 12 wherein there is a means of restraining the glove from sliding distally during its use.

19. The hand rehabilitation device of claim 18 wherein the restraining member is a strap securely fixed to the glove and having a mating loop and hook fastener to secure its closure around the wearer's hand or wrist.

20. The hand rehabilitation device of claim 12 wherein the elastic component may be replaced with a static or static progressive component.
